(19) United States
(12) Patent Application Publication
(10) Pub. No.: US 2015/0028228 A1
(43) Pub. Date: Jan. 29, 2015

(54) BELT TREATMENT SYSTEM

(71) Applicant: Vioguard LLC, Kirkland, WA (US)

(72) Inventors: Dale Almasy, Bellevue, WA (US); Roger Pangan, Kirkland, WA (US); Laurence Ranta, Seattle, WA (US); Eric Verme, Kirkland, WA (US)

(73) Assignee: Vioguard LLC, Kirkland, WA (US)

(21) Appl. No.: 13/950,902

(22) Filed: Jul. 25, 2013

(51) Int. Cl. B66B 31/02; A61L 2/10

(52) U.S. Cl. CPC B66B 31/02; A61L 2/10
USPC 250/492.1

(57) ABSTRACT

A handrail sanitization system incorporates ultraviolet light treatment into long straight sections of the handrail run, most preferably along the hidden return area of the endless loop forming the moving handrail. This treatment area allows for longer exposure times to the UV light and facilitates the treatment of the entire outer surface of the handrail. In some versions, an additional dosage controller is provided.

### FIELD OF THE INVENTION

This invention relates generally to ultraviolet sanitizing systems, and more particularly for such systems in use with escalator handrails.

### BACKGROUND OF THE INVENTION

There are numerous surfaces that are continually touched by a large number of people each day. Within airports, department stores, and other large buildings, escalator handrails are grasped by hundreds or even thousands of people on a daily basis. Indeed, because the moving walkway or escalator is an unstable surface the majority of those using the walkway or escalator will also grasp at least some portion of the handrail for stability. With a large number of people touching a common surface every day, it is exceedingly difficult to prevent germs from spreading to everyone who comes into contact with the handrail. This problem is compounded in airports because people are passing through from large geographic areas, bringing germs with them and thereby passing them along to others.

There have been a few attempts to automate the process of sanitizing escalator handrails, but all of them fail in one way or another. U.S. Pat. No. 7,134,539 describes a “guard device” for a handrail that includes a sterilization unit that includes a combination of ultraviolet lamps and an antibacterial spray. The '539 patent teaches that the guard device is to be installed in the entry zone of the escalator, with the sterilization system contained within the guard device. As such, the sterilization unit is positioned at the location where the handrail is curved, forming its hairpin turn prior to beginning the long straight run along the stairs or walkway. While this location may be convenient for access, it constrains the nature of the sanitation system and renders it only marginally effective. Indeed, the '539 patent describes a system that cannot sanitize the entirety of the handrail (including its upper and lower grasping surfaces) and provides for a limited UV exposure that may be less than desired or even ineffective.

A subsequent patent publication, number 2011/0158862, describes a similar system for handrail sterilization. The '862 publication further teaches that the sterilization system is to be incorporated in the return section of the handrail, combining a chemical spray with brushes and ultraviolet lights. Yet again, the UV lights are only directed toward a top surface of the handrail and provide little or no treatment of the side and bottom surfaces. In the '862 application the UV treatment is essentially an afterthought as the publication describes the chemical spray unit as being the primary sterilization unit and the UV region as being secondary. The use of the chemical treatment further requires the use of heat or other drying units to dry the liquid disinfectant before it emerges from the covered region so that users do not grasp a wet handrail and spread the disinfectant chemicals beyond the treating area. In addition, the disinfecting chemicals often contain powerful reducing agents, are harsh on the skin and may trigger reactions in sensitive individuals.

In general, prior art systems for sanitizing handrails have made an ineffective use of ultraviolet light and require the use of expensive and messy toxic chemicals in order to enhance the ability to sanitize the handrail.

### SUMMARY OF THE INVENTION

The preferred handrail sanitization system in accordance with the present invention incorporates ultraviolet light treatment into long straight sections of the handrail run, most preferably along the hidden return area of the endless loop forming the moving handrail. This treatment area allows for longer exposure times to the UV light and facilitates the treatment of the entire outer surface of the handrail.

In accordance with some versions of the invention, a plurality of treatment regions are provided in a return area of the handrail.

In preferred versions of the invention, multiple bulbs are provided, including UV bulbs directed toward the top, sides, and lower surfaces of the handrail.

In some versions of the invention, the UV treatment system is encased in a housing having internally reflective surfaces to direct the UV light inward and toward the handrail.

The preferred treatment system further includes at least one brush to wipe away extraneous dust or other matter prior to entering the treatment region.

### BRIEF DESCRIPTION OF THE DRAWINGS

Preferred and alternative examples of the present invention are described in detail below with reference to the following drawings:

FIG. 1 is a representative side view of an escalator handrail, shown with a plurality of treatment systems installed.

FIG. 2 is a perspective view of a preferred UV treatment system, shown with a handrail entering and emerging from the treatment system.

FIG. 3 is a sectional view of the treatment system of FIG. 2, taken along line A-A of FIG. 2.

FIG. 4 is an exploded view of an alternate treatment system for use with a moving handrail.

FIG. 5 is a perspective partial cutaway view of the treatment system of FIG. 4.

FIG. 6 is a block diagram of a UV treatment controller in accordance with preferred versions of the invention.

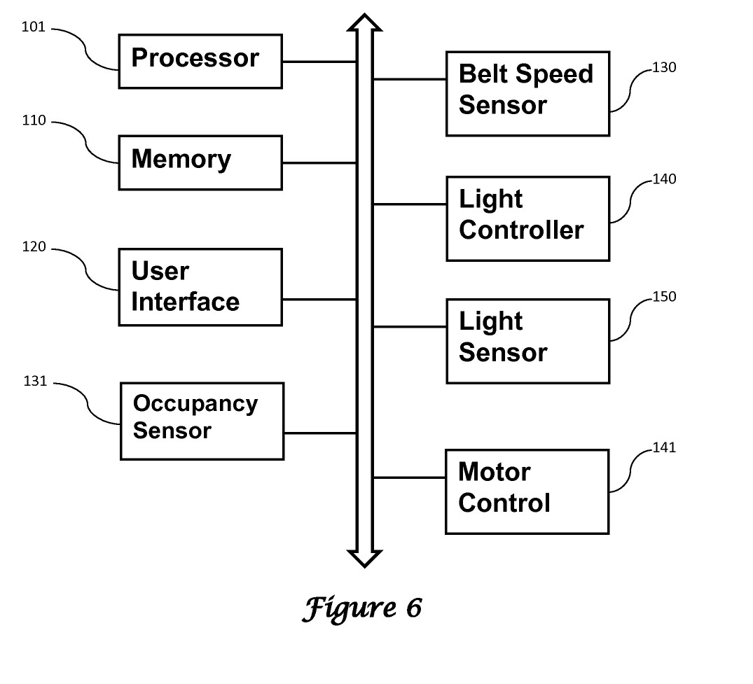

### DETAILED DESCRIPTION OF THE PREFERRED EMBODIMENT

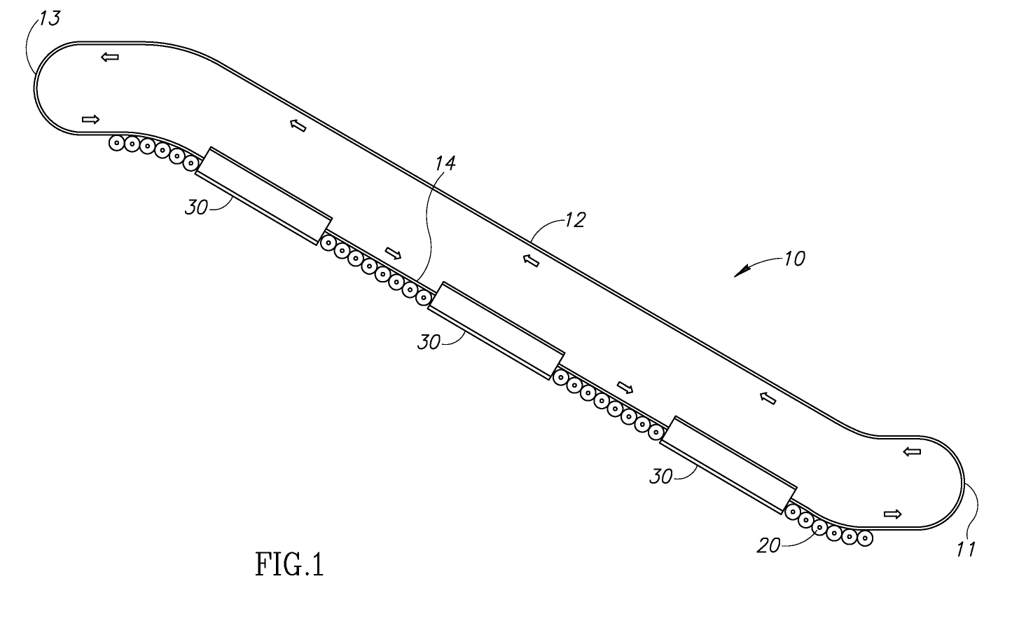

With reference to FIG. 1, a preferred version of the present invention is shown incorporated into a handrail for an escalator. In general, the treatment system is configured to be incorporated into a system having an endless loop type of surface, such as an escalator, moving walkway, moving handrail, or conveyor belt. In the illustrated version, the system includes a handrail 10 formed as an endless loop.

In a typical handrail configured with an escalator (or moving walkway) as illustrated in FIG. 1, the handrail 10 includes an emerging region 11, a functional or grasping region 12, a retracting region 13, and a return region 14. The handrail 10 is typically curved in the emerging region 11 as it changes direction from the return to begin its travel along the walkway or escalator stairway. In different implementations the handrail may be covered for much or at least a portion of the emerging region, or may be exposed for a majority or all the emerging region, as desired. After the emerging region the handrail follows a path defined by a grasping region 12, and in most cases the grasping region is defined by a relatively long, straight path. For example, in the illustrated version the majority of the grasping region is defined by a straight path from one end of the escalator run to the other end of the escalator run. At the end of the run, the handrail path of travel changes direction at the retracting region 13, which forms a hairpin curve so that the handrail may reverse course and return again to the emerging region. Between the retracting region and emerging region the handrail travels along a return region 14, which most efficiently defines a straight or relatively straight path of travel between these two points.

Although illustrated and described above with respect to a handrail for an escalator, a moving handrail follows a similar path for a moving walkway in which there is a grasping region and a return region, with emerging and retracting regions defined between the grasping and return regions. In the case of a conveyor belt or other form of endless loop, there is commonly an exposed area defining a functional path of travel and a hidden or return area defining a return path of travel.

Returning to FIG. 1, one or more treatment areas 30 are provided about the handrail 10, and as illustrated the treatment areas are positioned within the return area of the handrail. In the preferred version of the invention the treatment systems are not only positioned in the return area, but are mounted such that the handrail follows a substantially straight path of travel within the treatment system. In this sense, the term “substantially straight” means that the handrail may include a minor deflection (owing to the natural flexibility of the handrail itself) or minor curvature forming an angle of ten degrees or less from the point of entry of the handrail into the treatment unit to the exit of the handrail from the treatment unit. Most preferably, however, the treatment unit defines a straight path of travel such that the entry and exit points are along a straight line.

A typical handrail system as shown in FIG. 1 further includes a plurality of rollers 20 positioned along the return region to help maintain the handrail in its proper position and guide it along the return path. One or more of the rollers may be coupled to a motor to provide a force for propelling the handrail along its defined path.

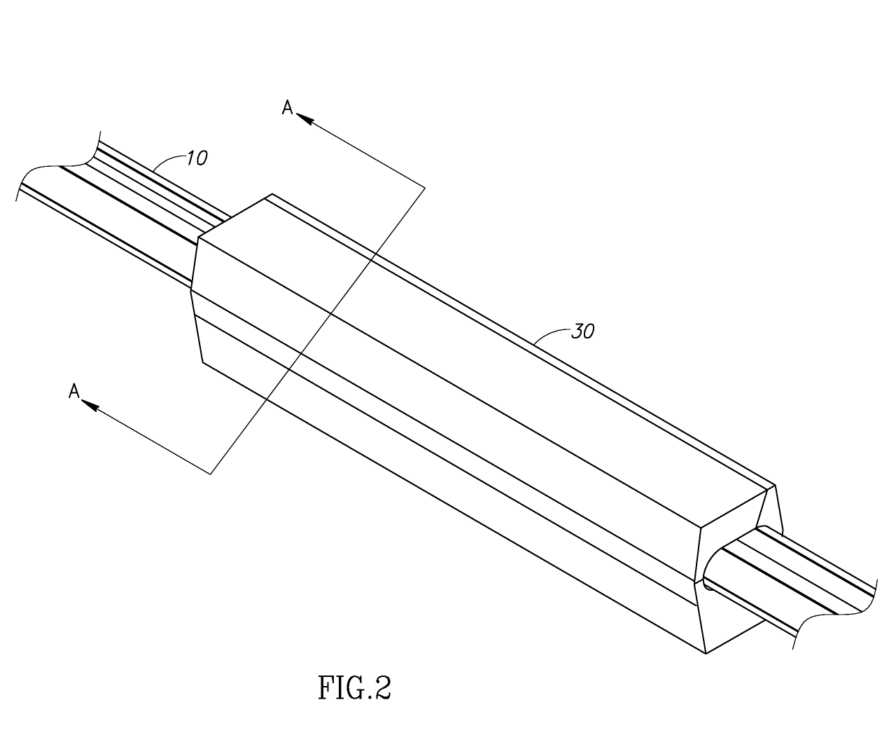

A preferred ultraviolet treatment system 30 is further illustrated in FIG. 2. As shown, a handrail 10 enters one end of the treatment system 30 and emerges from the other end. Within the outer case housing the treatment system the handrail is exposed to UV light.

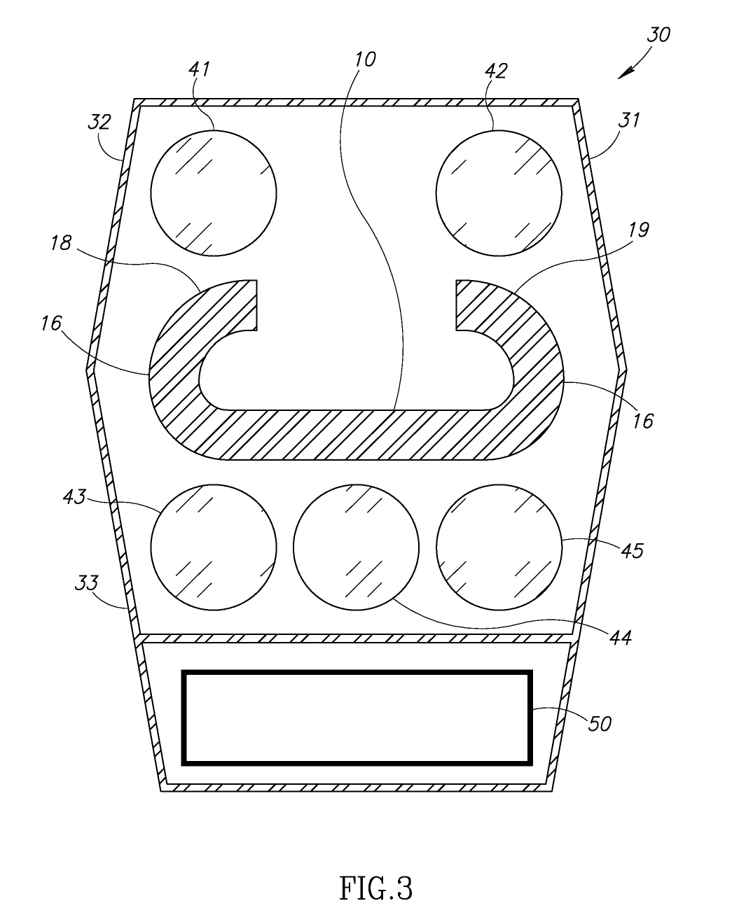

FIG. 3 provides a sectional view of a preferred treatment system 30 as shown in FIG. 2, taken along line A-A in FIG. 2. As shown, the treatment system is contained within an outer housing 31 defining an interior space for treating a handrail 10 with UV light. Within the interior space a plurality of bulbs 41-45 are provided, and in the illustrated version there are three lower bulbs 43, 44, 45 and two upper bulbs 41, 42.

A typical handrail for use with an escalator or moving walkway forms a C-shape in cross section, with the C being turned on its side. In the sectional view of FIG. 3 the handrail is shown from a position along its return path, and therefore it is upside-down from the orientation as perceived by a user who grasps the handrail along the grasping portion of the path of travel. From the orientation in FIG. 3, the handrail includes a bottom portion 15 positioned between a pair of side portions 16, 17. The side portions are preferably rounded along their outer surface, to provide a smooth surface for grasping, but otherwise the side portions extend generally vertically as they transition from the horizontal bottom portion. Each of the side portions transition to a top portion 18, 19 that are turned inward toward one another. In the typical implementation the top portions 18, 19 extend at least briefly along a line that is parallel to a plane defined by the bottom portion 1 transition to a top portion 18, 19 that are turned inward toward one another. In the typical implementation the top portions 18, 19 extend at least briefly along a line that is parallel to a plane defined by the bottom portion 15.

When the handrail 10 is positioned in the orientation occupied in the grasping region 12 of the escalator or moving walkway, a user grasping the handrail will typically hold the handrail such that the bottom portion 15 is in contact with the majority of the palm of the user's hand while a first side portion 17 and top portion 18 will be in contact with the thumb and the opposite second side portion 16 and top portion 19 will be in contact with the fingers and fingertips.

In the version of FIG. 3, three UV bulbs 43, 44, 45 are provided such that they direct light toward the bottom portion 15 of the handrail. A first upper UV bulb 41 is positioned to direct light directly upon a first upper portion 18 of the handrail, while a second upper UV bulb 42 is positioned to direct light directly upon a second upper portion 19 of the handrail. In this configuration, UV light shines directly on the top and bottom portions of the handrail 10.

The housing 31 preferably includes an upper sidewall 32 and a lower sidewall 33, with the upper and lower sidewalls being angled with respect to one another. The upper and lower sidewalls are joined together at an apex that forms an obtuse angle of less than 180 degrees, with the apex preferably being positioned at the middle of the side portions. The sidewalls are further formed with reflective surfaces on the interior of the sidewalls to reflect the UV light inward. Accordingly, the reflective surface and angular orientation reflects light from the upper and lower UV bulbs from the sidewalls and toward the side portions of the handrail.

In this configuration, a UV bulb is not positioned along the side of the handrail to shine light directly at the side portion of the handrail. A lower bulb alone would not provide sufficient light to provide proper treatment of either the top or side portions of the handrail. But the inclusion of both top and bottom bulbs, together with the angled housing sidewalls and reflective surface, provides an effective amount of light to be directed toward the side portion of the handrail.

A ballast and additional electrical components 50 may be mounted within the housing, and in the preferred version the housing forms a lower compartment containing these components. In other versions the ballast may be contained in an end or in a different portion of the housing.

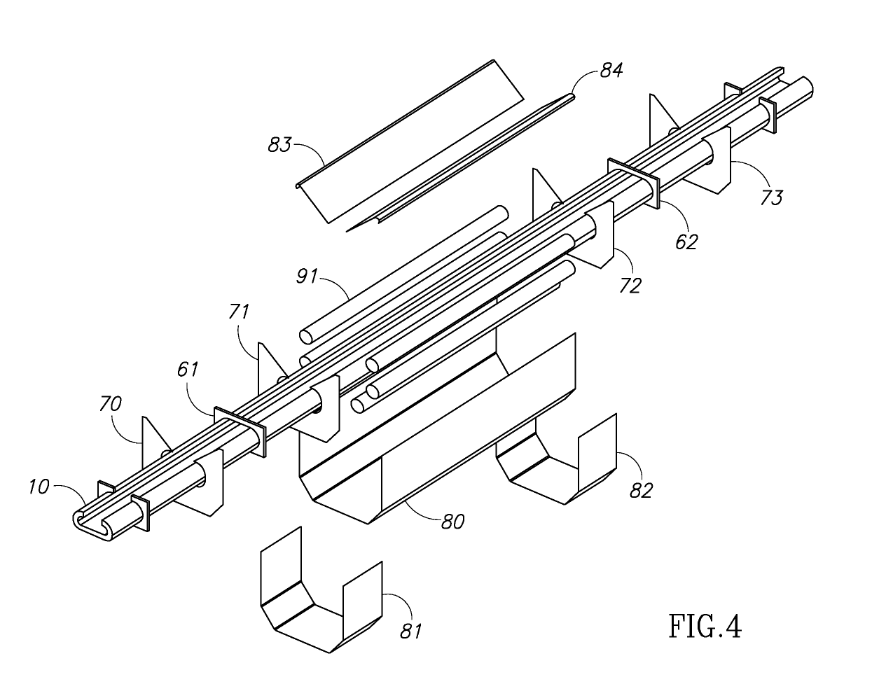

FIG. 4 provides a partial exploded view of an alternate version of the invention. As with FIGS. 2 and 3, the version of FIG. 4 is shown in its orientation along the return path of the handrail. In this version, a portion of a handrail 10 is shown entering and exiting a housing 80, although in this case the housing includes a pair of opposing sides that are generally straight, or vertical, rather than angled as shown in FIG. 3. The housing includes a first and second mounting bracket 81, 82, securing the housing to first and second end walls 71, 72. The end walls include openings that are sized to allow the handrail to enter and exit the housing, and preferably the openings are sized to allow the handrail to freely pass but with minimal clearance. Housing covers 83, 84 enclose the top portion of the housing. As with the version of FIG. 3, preferably the housing and covers are formed with a reflective surface on the interior of the housing and covers.

A preferred system may further include one or more brushes 61, 62, and preferably at least one brush is positioned upstream of the housing so that the handrail will pass along the brush before passing through the opening of the end wall and entering the housing 80. In the version as shown in FIG. 4 a brush is shown on each side of the housing, thereby allowing the escalator and handrail to travel in either direction and still include a brush upstream of the opening. At least one inlet protector 70, 71, 72, 73 is also positioned on either side of the opening, with the inlet protector providing a small passageway allowing entry of the handrail but blocking any other objects that may be attached to the handrail. Accordingly, the inlet protectors prevent debris from entering and damaging the UV lights and components within the housing. As with the brushes, inlet protectors are provided at each side of the housing.

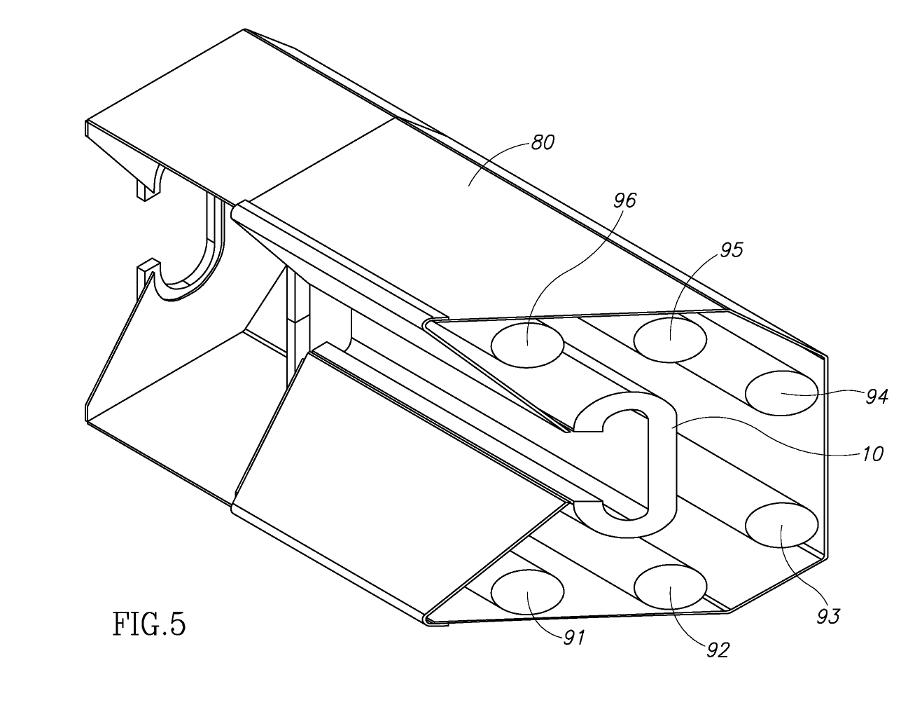

While the version of FIG. 3 included lights positioned along the top and bottom of the handrail but not the sides, the version of FIG. 4 incorporates UV lights positioned to shine directly upon the top, bottom, and both sides of the handrail. The position of the lights in the example of FIG. 4 is best seen with reference to FIG. 5, which shows a partial cutaway illustration of the same version of the invention. Thus, FIG. 5 illustrates a housing 80 with housing covers and a handrail moving through the interior space defined by the housing.

In the version of FIGS. 4 and 5, UV lights are positioned to shine light directly at the top, side, and bottom surfaces of the handrail. Accordingly, a pair of opposing top UV bulbs 91, 96 are positioned to direct light upon the top portion of the handrail 10, a pair of side UV lights 92, 95 are positioned to direct light upon the side portion of the handrail, and a pair of bottom UV lights 93, 94 are positioned to direct light upon the bottom portion of the handrail. Additional UV light bulbs may also be used, noting that in this version bulbs are positioned to shine light directly upon each of the surfaces of the handrail.

In accordance with preferred versions of the invention as described above, the UV light bulbs are preferably of the tube variety. This configuration of the housing and bulb type allows the housing to extend along lengthy sections of the handrail of 18 inches, 24, inches, 36 inches, 48 inches or more, depending on bulb availability. Thus, in accordance with one version of the invention UV tube lights can be positioned to direct light toward the handrail along all or the majority of the return path of the handrail. In this configuration the system provides a much longer treatment time for far greater effectiveness.

In some versions of the invention, one or more UV treatment systems are installed in a passive fashion, configured to shine UV light upon a handrail, conveyor belt, or other moving surface as described above. In other versions, a controller is provided to monitor and adjust the intensity of light in order to ensure ideal treatment dosage.

Because a typical handrail is in use during the day and moves at a constant speed, it is generally impractical to alter the time during which the handrail is exposed to UV light from within a single housing. In the course of designing a preferred system as described above, a key benefit is that dosage times can be controlled to an extent by incorporating an exposure length along the return path. In some instances, however, there may be a limit to the ability to lengthen or shorten the exposure distance, and in most cases the bulbs will vary in their effectiveness over time, and will burn out at an unexpected time. The incorporation of a preferred controller aids in maintaining desired dosages even in the presence of variable such as light intensity and exposure distances.

In some instances, escalators and moving sidewalks are configured to stop in order to conserve energy when no one is using them. In such an implementation a preferred sanitizing system likewise would be switched off and on from the AC mains or alternately have an enable/disable logic signal which switches on and off the UV lamps when the walkway is not in use. In one version the on/off control is implemented through an occupancy sensor 131 that may be, for example, an infrared beam or motion sensor positioned at the start of the walkway. Alternately this function could be implemented with a belt speed sensor 130 in communication with the belt and a controller. In either case, the system preferably turns off the UV lamps when the belt is not moving (as indicated by the belt speed being zero or the lack of an occupant, for example).

In one version, the controller includes a microprocessor 101 and an associated memory 110, the memory containing stored programming instructions operable by the processor to control the system in the fashion described further below. A user interface 120 is optionally provided, allowing for input and output to the controller. In one version the user interface includes a display screen and an input device such as a keyboard. In an alternate version the input device is a touchscreen that is also the display screen. Most preferably, the user interface allows a user to enter variables such as the overall length of the housing or to set preferred dosage levels for the system, as well as to receive an audio or visual indicator of the status of the system such as a burned out bulb or other event. Accordingly, the user interface may be located in an accessible housing in the vicinity of the escalator (or other device) or may be maintained in a remote control area such that the interface is in wired or wireless communication with the processor.

In a preferred version, the processor 101 is further in communication with a belt speed sensor 130, a light controller 140, and a light sensor 150. Where a belt speed sensor is included, it monitors the speed of the belt (or handrail) in order to derive the total time the belt or handrail is exposed to the UV light. In such a case, the bulb or housing length is a stored variable, maintained within the memory 110, so that the processor can determine exposure time as a function of housing length and belt travel speed. In other versions a belt speed sensor may be omitted and the exposure time may be a user-entered or pre-stored variable rather than one that is calculated as a function of belt speed.

The light sensor 150 detects the intensity of the UV light, and in a preferred version of the invention the light sensor is mounted within the housing and positioned to receive UV light directly from the bulbs. Most preferably several sensors are provided within the housing, positioned at multiple locations about the housing in order to detect the intensity of light from each of the separate bulbs within the housing. As the sensors are coupled to the processor, the processor determines the overall intensity of light being applied to each of the surfaces of the handrail, and likewise the total UV dosage as a function of intensity and exposure time.

The memory further contains instructions operable by the processor to allow the processor to control the intensity of the light, via the light controller 140, in order to control the dosage applied to the handrail. Thus, the processor is able to cause the light controller to increase or decrease the current to the bulbs to thereby increase or decrease the intensity. In some instances the length of the bulbs may be longer than necessary to provide an efficient treatment exposure, and in such cases the light controller 140 may decrease the current and thereby decrease the light intensity for greater overall efficiency. As a bulb ages over time, or in the event a particular bulb has burned out, the current may be increased to other bulbs in order to accommodate for the reduction in light intensity within the housing.

In a preferred version, the controller further indicates if a bulb has burned out, such as by sensing a corresponding drop in the light intensity or through other standard components for detecting a burned out bulb. For example, the controller may monitor the current delivered to the bulbs and detect when little or no current is flowing, thereby indicating that a bulb has burned out. In addition, the controller preferably tracks total usage time and total current applied to individual bulbs within the housing so that replacement times can be anticipated. In this configuration, the memory tracks such parameters and the processor causes the user interface to present an audible or visual indicator that a bulb is due for replacement. Varying the lamp intensity will generally result in shorter life, so by allowing intensity to be controlled, the user can trade off pathogen kill efficacy with bulb life.

The dosage levels may be user-controlled or preset within the memory, according to preferred dosages to apply to the handrail. For example, the greatest dosage expected to be required for a 100 percent kill dosage for bacteria is approximately 46,000 microwatt-sec/square cm. The user interface controls may provide for a “100 percent bacteria” setting, under which the controller will seek to increase light intensity to produce the desired dosage (or to the nearest the system can achieve to the desired dosage in the event the exposure time and intensity cannot obtain the 100 percent level). The controls may also provide for a “90 percent bacteria” setting, in which the bulbs operate at an intensity sufficient to kill 90 percent of all bacteria. Other settings may be provided for molds, viruses, or other pathogens specifically, and may allow for either 100 percent kill dosages or a desired percentage other than 100 percent. An additional setting may indicate a dosage for all known pathogens, and another setting for high intensity disinfection in which the device operates for a duration beyond that which is believed to be a necessary dosage to kill all known pathogens.

In one preferred implementation, the device includes a downtime cleaning cycle intended to kill 100 percent of all bacteria. In this setting, in the event the normal use of the system includes a belt speed that is too fast and results in an exposure time that is too small to obtain a 100 percent kill dosage, the belt speed may be slowed to increase dosage in order to provide an occasional sanitizing of the belt. Most preferably, this cycle would be used overnight or during other down times when people are not on the escalator or walkway. In one version, a control switch (for example, in the form of the user interface 120) is operable and in communication with the processor to initiate the deep cleaning cycle. In response to initiation of such a cycle, a speed control signal (output as a motor control signal 141) is provided to the motor drive electronics of the escalator or moving walkway to cause it to operate at a speed sufficient for the deep cleaning cycle. Once the deep cleaning cycle is complete the system returns to normal operation. It should be appreciated that an absolute 100% kill is statistically unlikely to be achieved with this type of device; instead microbiologists speak of a “log 4 reduction” or a “log 5 reduction”, for example, which means 99.99% and 99.999% kill, respectively.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A sanitation device for an endless belt configured to travel through a functional region and a return region, the sanitation device comprising:
a housing defining an interior space, the housing configured to receive a portion of the belt within the interior space, the housing further being positioned within the return region; and
an ultraviolet light source contained within the housing and positioned to direct light toward the portion of the belt within the interior space

2. The sanitation device of claim 1, wherein the ultraviolet light source comprises a plurality of elongated light bulbs.

3. The sanitation device of claim 1, wherein the housing further comprises a first end configured to allow the belt to enter the housing and a second end configured to allow the belt to exit the housing, the housing being substantially straight from the first end to the second end.

4. The sanitation device of claim 3, wherein the housing is straight from the first end to the second end.

5. The sanitation device of claim 3, wherein the ultraviolet light source comprises a plurality of elongated light bulbs, the light bulbs being 24 inches or greater in length.

6. The sanitation device of claim 3, wherein an interior surface of the housing is reflective.

7. The sanitation device of claim 3, wherein the belt comprises a handrail having a cross-sectional shape as viewed in the return region that includes a top portion, a pair of opposing side portions, and a bottom portion, and further wherein the ultraviolet light source is configured to directly illuminate the top portion and the bottom portion.

8. The sanitation device of claim 7, wherein the housing comprises a sidewall forming an angle of less than 180 degrees, the apex of the angle being positioned between the top portion and the bottom portion of the sidewall to reflect light toward at least one of the opposing side portions.

9. The sanitation device of claim 7, wherein the ultraviolet light is further configured to directly illuminate each of the opposing side portions.

10. The sanitation device of claim 3, further comprising a controller, the controller having a process and a memory containing stored programming instructions operable by the processor to control a dosage of ultraviolet light applied by the ultraviolet light source to the belt.

11. The sanitation device of claim 10, further comprising:
a light sensor positioned to detect light emitted from the ultraviolet light source, the light sensor being in communication with the processor;
a light controller in communication with the processor, the memory containing stored programming instructions operable by the processor to cause the light controller to adjust the current delivered to the ultraviolet light source to control the dosage of ultraviolet light applied by the ultraviolet light source to the belt.

12. The sanitation device of claim 11, further comprising a belt speed sensor configured to determine a speed of travel of the belt, the belt speed sensor being in communication with the processor, the memory further containing stored programming instructions operable by the processor to cause the light controller to adjust the current delivered to the ultraviolet light source to control the dosage of ultraviolet light applied by the ultraviolet light source to the belt, wherein the dosage is a function of the speed of travel of the belt and the intensity of the ultraviolet light.

13. The sanitation device of claim 11, further comprising a user interface, the memory further having stored programming instructions operable by the processor to cause the user interface to display an indicator related to a parameter of the dosage of applied ultraviolet light.

14. The sanitation device of claim 11, further comprising a user interface, the memory further having stored programming instructions operable by the processor to receive an input via the user interface, the input comprising a user-settable dosage level, whereby the stored programming instructions operable by the processor cause the light controller to adjust the current delivered to the ultraviolet light source to control the dosage of ultraviolet light applied by the ultraviolet light source to the belt as a function of the input.

15. A sanitation system, comprising a plurality of sanitation devices in accordance with claim 1, the housing of each of the plurality of sanitation devices being positioned within the return region.
